(19) United States
(12) Patent Application Publication
(10) Pub. No.: US 2013/0190603 A1
(43) Pub. Date: Jul. 25, 2013

(54) METHOD FOR ULTRASOUND FOCAL SPOT SHAPING

(71) Applicants: Rares Salomir, Ambilly (FR); Magalie Viallon, Lyon (FR)

(72) Inventors: Rares Salomir, Ambilly (FR); Magalie Viallon, Lyon (FR)

(21) Appl. No.: 13/355,723

(22) Filed: Jan. 23, 2012

(51) Int. Cl. A61B 5/055; A61N 7/00

(52) U.S. Cl. 600/411; 601/2

(57) ABSTRACT

The focus of a focused ultrasound system is calibrated with respect to a frame-of-reference, such as that of a magnetic resonance system, by selectively activating only specific regions of the ultrasound transducer of the ultrasound system, thereby altering the shape of the focus. The focal shape is selectively modified in shape and positioned in successive steps of a calibration procedure, while moving the focus through successive localizers of the frame-of-reference. The parameters for operating the ultrasound system when the calibration procedure is completed are stored as parameters that accurately position the focus of the ultrasound system with respect to the frame-of-reference.

### BACKGROUND OF THE INVENTION

1. Field of the Invention

The present invention concerns a method for shaping a focal spot of a focused ultrasound system, and in particular to focusing such a focal spot with respect to the frame-of-reference of a magnetic resonance system.

2. Description of the Prior Art

When using magnetic resonance (MR) for image guidance of focused ultrasound, it is standard practice to calibrate the frame-of-reference of the focused ultrasound system with respect to the frame-of-reference of the MR system. This involves firing low power test shots from the ultrasound array, and generating images of the influence of these test shots on a phantom with magnetic resonance techniques such as MR thermometry or MR radiation-force imaging.

As is typical for MR data acquisitions, in this context there exists tradeoffs between the speed, resolution and the volume that is evaluated (probed). This stands in conflict with the desire to determine the position of the test sonication quickly, accurately and robustly, i.e., without the sonicated region being outside of the examination volume.

In practice, therefore, a reasonable scan rate for the magnetic resonance data acquisition is achieved by choosing a reasonable resolution, and limiting the field of view of the MR data acquisition apparatus.

### SUMMARY OF THE INVENTION

An object of the present invention is to provide a method for shaping the focal spot of an ultrasound transducer array with respect to a frame-of-reference, in particular to the frame-of-reference of an MR system.

It is a further object of the present invention to provide such a method wherein, if the field of view of the MR system is limited, the test shots emitted by the ultrasound array that are used in the calibration procedure will be inside the probed volume.

It is a further object of the present invention to provide such a method wherein the determined position of the focal spot provides high-resolution spatial information.

As noted above, in conventional calibration of the focal spot of an ultrasound system to the frame-of-reference of an imaging system, such as an MR system, the volume coverage, including the slice thickness, is chosen to be sufficiently large so as to ensure that the test sonication position is detected. Acquiring MR data from a larger volume requires a longer amount of time, and therefore the conventional selection of a relatively large volume coverage unavoidably takes place at the expense of time or resolution. In accordance with the invention, the above objects are achieved by a method for shaping the focus of an ultrasound array or transducer, by activating only specific regions of the transducer/array, so that the shape of the focus (focal shape) is selectively altered according to the activated regions. By intentionally modifying the focal shape and position during several steps in a calibration procedure, information with high-spatial resolution can be obtained while ensuring that the positions are found within a few number of scans.

### BRIEF DESCRIPTION OF THE DRAWINGS

FIGS. 1, 2, 3 and 4 schematically illustrate successive steps in an embodiment of the method according to the invention.

### DESCRIPTION OF THE PREFERRED EMBODIMENTS

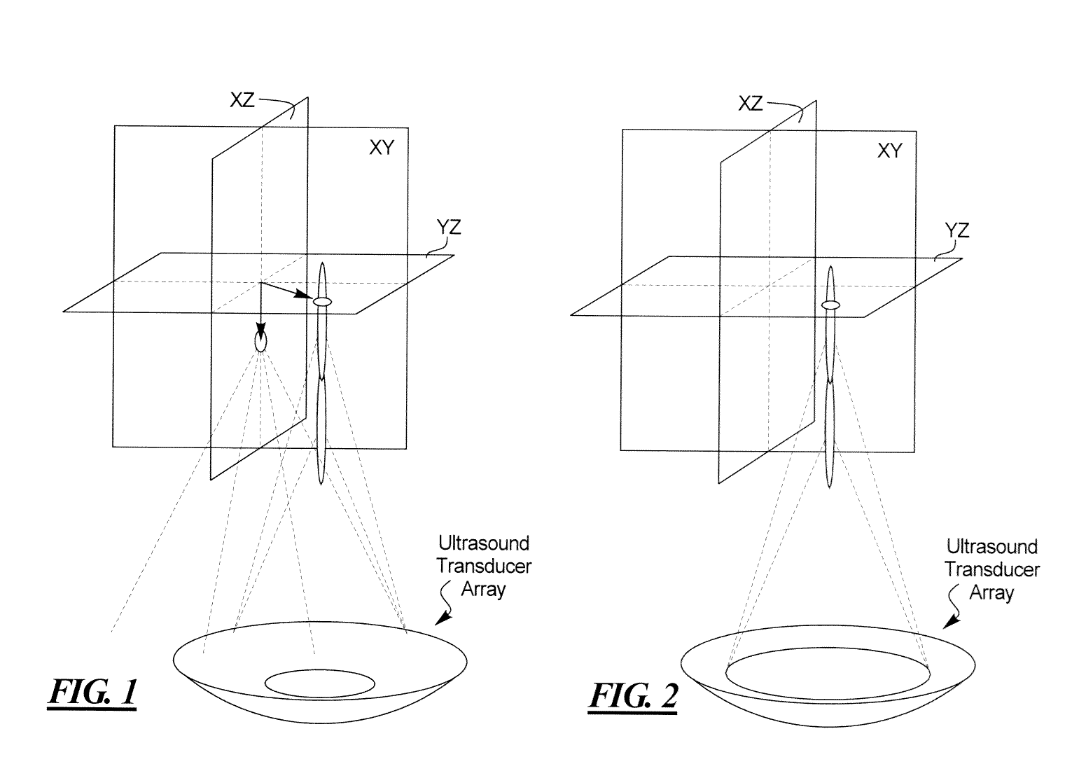

In each of FIGS. 1, 2, 3 and 4, three orthogonal localizers are schematically indicated. In the exemplary embodiment shown in the drawings, these localizers are respective planes YZ, XY and XZ in a Cartesian coordinate system, but any set of appropriate localizers can be used that define or describe a frame-of-reference in which ultrasound emitted by an ultrasound transducer array is to be utilized, and with respect to which the position of the focus of the ultrasound array must be known.

The localizers may, for example, define the frame-of-reference of a magnetic resonance system.

As is known, predominantly activating the central elements of the ultrasound transducer array will result in a focus having a well-defined central axis, but the focus will be elongated along this central axis. Activating predominantly the peripheral regions of the array results in a focus that is longitudinally very short, but slightly wider (i.e., with a larger spread in a plane perpendicular to the longitudinal axis.

In the exemplary embodiment shown in the drawings, the long focus is used to first localize a point along the symmetry axis of the ultrasound transducer array. Because this focus is very narrow, the center of this focus can be determined with high accuracy. Determining the center and shape of such a focus can be done in any number of suitable ways, such as by sonicating a phantom and obtaining MR data from the sonicated phantom and processing the MR data by MR thermometry or MR radiation-force imaging.

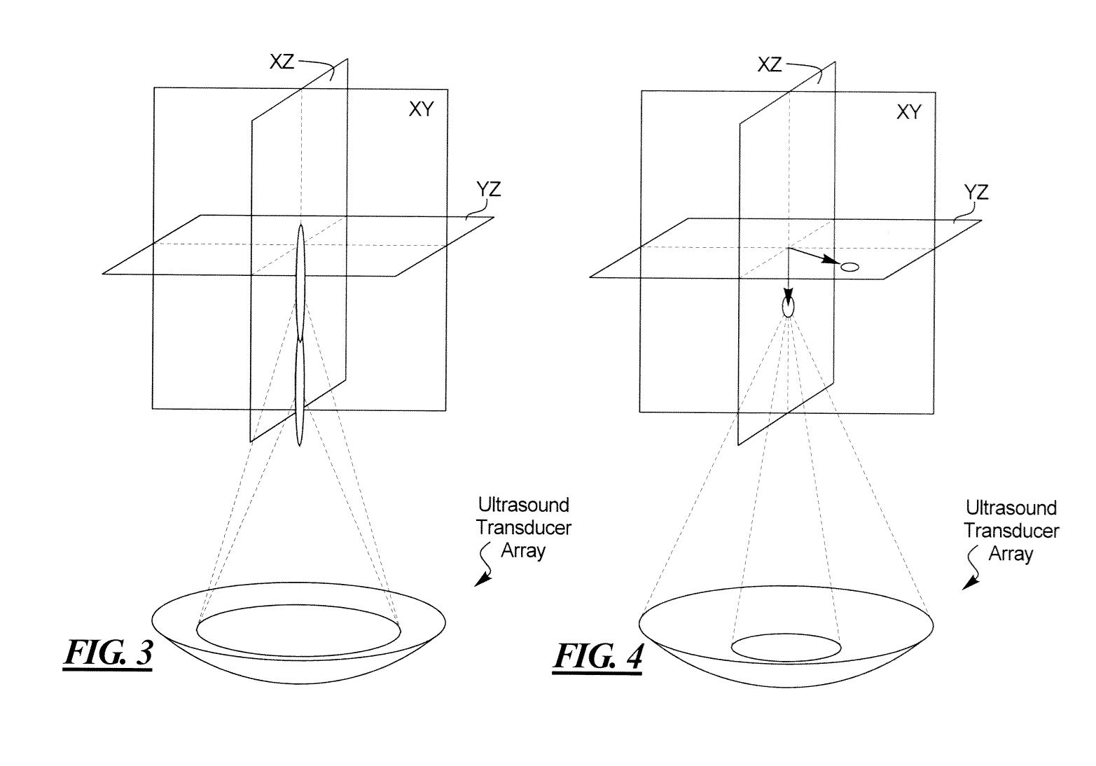

The ultrasound transducer array is then shifted, either physically or by electronic adjustment of the respective phases of the activating signals supplied to the individual elements of the transducer array, such that two of the localizer planes now contain the new focal point, and such that their intersecting axes are also the axis of the transducer. This is shown in FIGS. 2 and 3. Testing or confirmation can be made in a further sonication with an elongated focal point. To determine the true center of the natural focus, a short-focus sonication may be used, as shown in FIG. 4.

It is of course also possible to shift the localizer planes instead of the transducer array.

A number of image processing techniques can be used. Centroids or Gaussian fits, particularly multi-dimensional Gaussian fits, may be used to detect the center of the test sonications. The results of the detected points may be used to define fixed registration parameters, but may also be used to identify curved or non-fixed mappings of the transducer position compared to the MR image space which, like the ultrasound system, may exhibit non-linear distortions.

For any system wherein a focus is generated from a set of solid angles, the extent to which the focus will be short (spatially well-defined) in one direction depends on the number of contributions to the focus from signals arriving from solid angles that are substantially orthogonal to that direction. For example, in the case of a naturally focused transducer, this means that if the transducers are fired only from below, relatively few contributions are made from the solid angles in the horizontal plane, therefore resulting in an elongation in the up-down direction. In the other extreme, in a system without a natural focus, such as a flat plate, the focus is established by choosing appropriate phases for the individual transducer elements. Again, solid angles that are closely aligned with a coronal plane determine the focus size in the interior/posterior direction, and those from an axial direction determine the focus definition in the head-to-toe direction. Those from a sagittal plane determine the definition in the left-right direction.

Although modifications and changes may be suggested by those skilled in the art, it is the intention of the inventors to embody within the patent warranted hereon all changes and modifications as reasonably and properly come within the scope of their contribution to the art.

1.-2. (canceled)

3. A method as claimed in claim 6 wherein said focused ultrasound system comprises a plurality of individually activatable transducer elements, each activated with a respective signal having a phase, and comprising shifting said focus in step (d) electronically by adjusting the respective phases with respect to each other.

4.-5. (canceled)

6. A method for alignment of a focal point of a focused ultrasound system, comprising:
(a) placing a subject in a frame of reference defined by a plurality of mutually intersecting localizers;
(b) operating said focused ultrasound system with an initial set of operating parameters to sonicate the subject with a focused ultrasound beam having a focal point surrounded by a focal spot that produces at least one region of measurable temperature elevation or a measurable acoustic radiation force signal in at least one of said localizers at a time, said focal spot having a shape that is determined by said initial set of operating parameters;
(c) changing said parameters of said focused ultrasound system to selectively modify the shape of the focal spot while moving the focal point through successive localizers in said frame of reference, resulting in said focused ultrasound system having a last set of operating parameters with said focal point in a last of said successive localizers; and
(d) calibrating said focused ultrasound system with respect to a predetermined target by setting said operating parameters of said ultrasound system to said last set of operating parameters from step (c).

7. A method as claimed in claim 1 comprising aligning said focal point of said focused ultrasound system on said predefined target by:
(e) visualizing an intersection of the focal spot at a first of said localizers;
(f) operating said focused ultrasound system to shift said ultrasound beam perpendicularly on a longitudinal axis of said ultrasound beam until the longitudinal axis of the ultrasound beam coincides with an axis of intersection of second of said localizers and third of said localizers; and
(g) shifting said focal point along said longitudinal axis until said focal point is at an intersection of said first of said localizers, said second of said localizers, and said third of said localizers.

8. A method for alignment of a focal point of a focused ultrasound system, comprising:
(a) placing a subject in a frame of reference defined by a plurality of mutually intersecting localizers;
(b) operating said focused ultrasound system with an initial set of operating parameters to sonicate the subject with a focused ultrasound beam having a focal point surrounded by a focal spot that produces at least one region of measurable temperature elevation or a measurable acoustic radiation force signal in at least one of said localizers at a time, said focal spot having a shape that is determined by said initial set of operating parameters;
(c) changing said parameters of said focused ultrasound system to selectively modify the shape of the focal spot while moving the focal point through successive localizers in said frame of reference, resulting in said focused ultrasound system having a last set of operating parameters with said focal point in a last of said successive localizers; and
(d) calibrating said focused ultrasound system with respect to a predetermined target by storing said operating parameters of said ultrasound system to said last set of operating parameters from step (c).

9. A method as claimed in claim 8 wherein said focused ultrasound system comprises a plurality of individually activatable transducer elements, each activated with a respective signal having a phase, and comprising shifting said focus in step (d) electronically by adjusting the respective phases with respect to each other.
